(19) United States
(12) Patent Application Publication
(10) Pub. No.: US 2018/0356415 A1
(43) Pub. Date: Dec. 13, 2018

(54) FLUOROUS COMPOUND, METHOD OF PREPARING FLUOROUS TAGGED PROTEIN, AND METHOD OF IMMOBILIZING PROTEIN

(71) Applicant: National Tsing Hua University, Hsinchu City (TW)

(72) Inventors: Chun-Cheng Lin, Taichung City (TW); Ben-Yuan Li, New Taipei City (TW)

(73) Assignee: National Tsing Hua University, Hsinchu City (TW)

(21) Appl. No.: 15/829,964

(22) Filed: Dec. 3, 2017

(30) Foreign Application Priority Data
Jun. 8, 2017 (TW) 106119084

(51) Int. Cl. G01N 33/543; C07K 5/062; C07F 5/02; C07K 5/072; C07C 323/25; C07K 14/245; C07K 14/435; C07K 16/18

(52) U.S. Cl. CPC G01N 33/54353; G01N 33/54393; C07K 5/0606; C07F 5/025; C07K 2319/24; C07C 323/25; C07K 14/245; C07K 14/43595; C07K 16/18; C07K 5/06095

(57) ABSTRACT

A fluorous compound, a method of preparing a fluorous tagged protein, and a method of immobilizing protein are provided. The fluorous compound is represented by Y-L-R, wherein Y is a fluorous group; L is a linker, and the linker includes a bivalent group having a sulfo group, a bivalent group having a carboxyl group, or a bivalent group of hydrophilic amino acid; and R is a functional group capable of bonding to protein.

### CROSS-REFERENCE TO RELATED APPLICATION

This application claims the priority benefit of Taiwan application serial no. 106119084, filed on Jun. 8, 2017. The entirety of the above-mentioned patent application is hereby incorporated by reference herein and made a part of this specification.

### BACKGROUND OF THE INVENTION

### Field of the Invention

The invention relates to a compound and a method of preparing tagged protein and immobilizing protein using tagged protein, and more particularly, to a fluorous compound, a method of preparing fluorous tagged protein, and a method of immobilizing protein using the fluorous tagged protein.

### Description of Related Art

The protein microarray is a very important tool in proteomics. The protein microarray performs a large amount of biochemical property and bioactivity analysis using protein immobilized on a solid support.

Currently, many protein immobilization methods have been developed. For instance, protein can be cured via random covalent attachment. However, this method has the drawback of non-specific orientation of protein on the solid support, and therefore immobilized protein activity may be significantly reduced.

Moreover, protein can also be immobilized on a chip using non-covalent attachment. However, this method has drawbacks such as potential non-specific adsorption between protein and the solid support and insufficient strength of non-covalent bond force. Non-specific protein adsorption on the solid support may reduce the carrying capacity of the target protein and interfere with analysis results. Moreover, insufficient strength of non-covalent attachment readily causes protein separation.

### SUMMARY OF THE INVENTION

The invention provides a fluorous compound having high solubility in an aqueous solution.

The invention provides a method of preparing a fluorous tagged protein having high fluorous tagging efficiency.

The invention provides a method of curing a protein having orientation specificity that can reduce protein non-specific adsorption.

The fluorous compound of the invention is represented by Y-L-R, wherein Y is a fluorous group; L is a linker, and the linker includes a bivalent group having a sulfo group, a bivalent group having a carboxyl group, or a bivalent group of hydrophilic amino acid; and R is a functional group capable of bonding to protein.

In an embodiment of the invention, the linker can further include a C1 to C12 alkylene group, a C6 to C15 arylene group, a C2 to C12 heteroarylene group, a C1 to C12 alkyleneoxy group, a C1 to C12 alkylene sulfide group, a C3 to C12 cycloalkylene group, an amide group, a bivalent group of ethylene glycol, or a combination thereof.

In an embodiment of the invention, the bivalent group having the sulfo group is, for instance, a bivalent group of cysteic acid.

In an embodiment of the invention, the bivalent group of the hydrophilic amino acid is, for instance, a bivalent group of aspartic acid, a bivalent group of glutamic acid, or a bivalent group of arginine.

In an embodiment of the invention, the fluorous group can include a straight or branched C3 to C8 fluoroalkyl group.

In an embodiment of the invention, the functional group capable of bonding to protein can include a group having a thiol group and an amine group, a group having a thioester group, or a group having a boric acid group.

In an embodiment of the invention, the group having the boric acid group is, for instance, a group represented by formula (1) below:

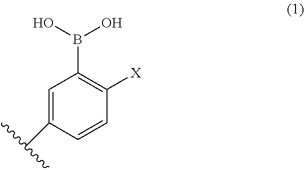

wherein X is hydrogen or a substituent containing at least one atom of nitrogen, oxygen, or sulfur.

In an embodiment of the invention, the fluorous compound is, for instance, a compound represented by formula (2) or a compound represented by formula (3) below:

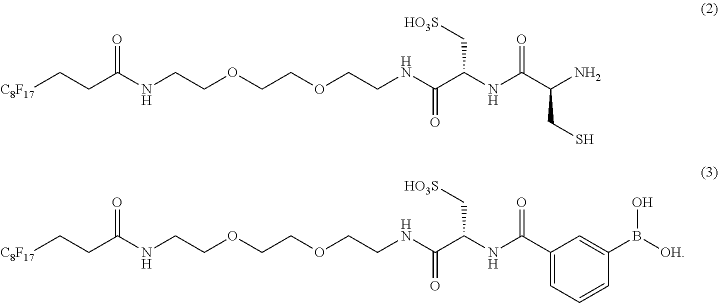

The method of preparing the fluorous tagged protein of the invention includes the following steps. A protein is provided. A fluorous compound is bonded to the protein, and the fluorous compound is represented by Y-L-R. Y is a fluorous group; L is a linker, and the linker includes a bivalent group having a sulfo group, a bivalent group having a carboxyl group, or a bivalent group of hydrophilic amino acid; and R is a functional group capable of bonding to protein.

In an embodiment of the invention, the linker can further include a C1 to C12 alkylene group, a C6 to C15 arylene group, a C2 to C12 heteroarylene group, a C1 to C12 alkyleneoxy group, a C1 to C12 alkylene sulfide group, a C3 to C12 cycloalkylene group, an amide group, a bivalent group of ethylene glycol, or a combination thereof.

In an embodiment of the invention, the hydrophilic amino acid is, for instance, a bivalent group of aspartic acid, a bivalent group of glutamic acid, or a bivalent group of arginine.

In an embodiment of the invention, the functional group capable of bonding to protein includes a group having a thiol group and an amine group, a group having a thioester group, or a group having a boric acid group.

In an embodiment of the invention, the group having the boric acid group is, for instance, a group represented by formula (1) below:

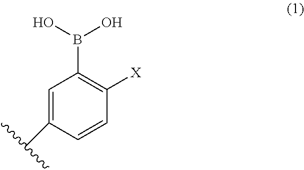

wherein X is hydrogen or a substituent containing at least one atom of nitrogen, oxygen, or sulfur.

In an embodiment of the invention, provided that the functional group capable of bonding to protein is the group having the thiol group and the amine group, the C terminal of the protein has a thioester group, and the protein and the fluorous compound can be bonded via native chemical ligation (NCL).

In an embodiment of the invention, the protein is, for instance, a recombinant protein expressed using an IMPACT™-CN protein expression system.

In an embodiment of the invention, provided that the functional group capable of bonding to protein is the group having the thioester group, the N terminal of the protein is cysteine, and the protein and the fluorous compound are bonded via NCL.

In an embodiment of the invention, provided that the functional group capable of bonding to protein is the group having the boric acid group, the protein includes a fragment crystallizable region (Fc region) having a sugar chain, the sugar chain includes a diol group, and the protein and the fluorous compound are bonded by boronate ester formation.

In an embodiment of the invention, the protein includes a glycosylated antibody or Fc-fusion protein.

The method of immobilizing protein of the invention includes the following steps. A fluorine-modified surface is provided. A fluorous tagged protein is brought in contact with the fluorine-modified surface, wherein the fluorous tagged protein is immobilized on the fluorine-modified surface via fluorous-fluorous interaction.

In an embodiment of the invention, the fluorine-modified surface is, for instance, the surface of a chip or the surface of a nanoparticle.

Based on the above, since the fluorous compound of the invention includes a hydrophilic group, the solubility of the fluorous compound to water is significantly improved. Moreover, the fluorous tagged protein is prepared using the fluorous compound of the invention, and therefore higher fluorous tagging efficiency is achieved. Moreover, since the method of protein immobilization of the invention can immobilize the fluorous tagged protein on the fluorine-modified surface via fluorous-fluorous interaction, orientation specificity can be achieved, and protein non-specific adsorption can be reduced.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a mass spectrum of purified protein and maltose binding protein MBP not tagged by fluorine.

FIG. 2 is an eGFP fluorescence analysis chart of the fluorous chips of experimental example 1 and comparative experimental example 1 immobilized by protein.

FIG. 3 is a Cy3 fluorescence analysis chart of the fluorous chips of experimental example 2, experimental example 3, and comparative experimental example 2 immobilized by protein.

FIG. 4 is a Cy3 fluorescence analysis chart of the fluorous chips of experimental example 4, comparative experimental example 3, and comparative experimental example 4 immobilized by protein.

### DESCRIPTION OF THE EMBODIMENTS

In the following, embodiments are provided to further describe the invention, but the embodiments are only exemplary and are not intended to limit the scope of the invention.

### [Fluorous Compound of the Invention]

The fluorous compound of an embodiment of the invention can be represented by Y-L-R, wherein Y is a fluorous group, L is a linker, and R is a functional group capable of bonding to protein. In the present embodiment, the linker L includes at least one hydrophilic group. The hydrophilic group is, for instance, a bivalent group having a sulfo group, a bivalent group having a carboxyl group, or a bivalent group of hydrophilic amino acid.

In an embodiment of the invention, the bivalent group having the sulfo group is, for instance, a bivalent group of cysteic acid. The bivalent group of hydrophilic amino acid includes, for instance, a bivalent group of aspartic acid, a bivalent group of glutamic acid, or a bivalent group of arginine.

Since the affinity of a regular fluorous compound to organic solvent and water is poor, the solubility of the regular fluorous compound in an aqueous solution is poor. However, in the present embodiment, since the linker L of the fluorous compound includes at least one hydrophilic group from the above, water solubility of the fluorous compound of the invention can be increased.

In an embodiment of the invention, the linker L can further include a C1 to C12 alkylene group, a C6 to C15 arylene group, a C2 to C12 heteroarylene group, a C1 to C12 alkyleneoxy group, a C1 to C12 alkylene sulfide group, a C3 to C12 cycloalkylene group, or a bivalent group of ethylene glycol, but the invention is not limited thereto.

In an embodiment of the invention, the fluorous group Y is, for instance, a straight or branched C3 to C8 fluoroalkyl group.

In an embodiment of the invention, a functional group R capable of bonding to protein can include a group having a thiol group and an amine group, a group having a thioester group, or a group having a boric acid group. The fluorous compound can be bonded to protein via the functional group to tag the protein with fluorine.

In an embodiment of the invention, the group having the boric acid group is a group represented by formula 1 below.

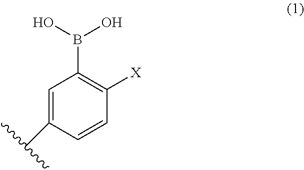

wherein X is hydrogen or a substituent containing at least one atom of nitrogen, oxygen, or sulfur. Since the group represented by formula 1 has a benzene ring, electrons adjacent to the boric acid can be stabilized.

In an embodiment of the invention, the fluorous compound is a compound represented by formula (2) or a compound represented by formula (3) below:

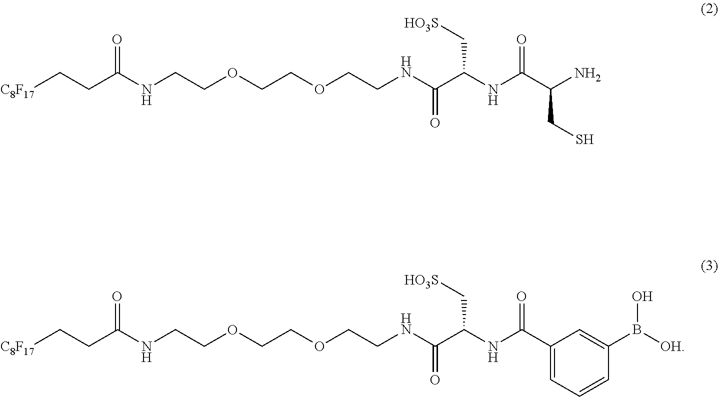

### [Method of Preparing Fluorous Tagged Protein of the Invention]

The method of preparing a fluorous tagged protein of the invention includes the following steps.

First, a protein is provided. The protein is, for instance, naturally occurring protein, antibody, or recombinant protein obtained by genetic engineering and protein engineering.

Next, the fluorine compound is bonded to the protein. The bonding of the fluorous compound and protein needs to occur in an aqueous solution, and therefore the water solubility of the fluorous compound has a significant impact on the bonding efficiency of the fluorous compound and protein. Since the linker L of the fluorous compound of the invention includes at least one hydrophilic group from the above, the issue of reduced bonding efficiency with protein caused by poor water solubility of the fluorous compound can be prevented.

In the present embodiment, the bonding between the fluorous compound and protein occurs via the functional group R of the fluorous compound capable of bonding to protein and the corresponding group of the target protein.

For instance, in an embodiment of the invention, when the functional group R of the fluorous compound capable of bonding to protein is the group having the thiol group and the amine group, the C terminal of the target protein has a thioester group, and the target protein and the fluorous compound are bonded via NCL.

Specifically, in the present embodiment, recombinant target protein is expressed by an IMPACT™-CN (Intein Mediated Purification with Affinity Chitin-binding Tag) protein expression system, wherein the C terminal of the recombinant target protein has a thioester group capable of reacting with the group having the thiol group and the amine group of the fluorous compound in an NCL reaction to generate fluorous tagged protein.

In another embodiment of the invention, when the functional group R of the fluorous compound capable of bonding to protein is the group having the thioester group, the N terminal of the target protein is cysteine, and the protein and the fluorous compound are bonded via NCL. Specifically, the cysteine of the N terminal of the target protein has a thiol group and an amine group and can react with the group having the thioester group of the fluorous compound in an NCL reaction to generate fluorous tagged protein.

In another embodiment of the invention, when the functional group R of the fluorous compound capable of bonding to protein is the group having the boric acid group, the target protein is, for instance, glycoprotein. In an embodiment, the target protein includes a fragment crystallizable (Fc) region having a sugar chain.

In the present embodiment, the target protein having the Fc region is, for instance, an antibody or Fc-fusion protein, wherein the Fc-fusion protein can be obtained by genetic engineering and protein engineering. The target protein includes a fragment crystallizable (Fc) region having a sugar chain, and the sugar chain includes a diol group. The diol group is, for instance, a vicinal diol group of the adjacent carbon atoms or a diol group of the other carbon atoms close to each other due to spatial distribution.

In the present embodiment, the target protein and the fluorous compound are bonded by boronate ester formation. Specifically, the diol group on the sugar chain of the target protein and the group having the boric acid group of the fluorous compound form boronate ester to bond the target protein and the fluorous compound to generate fluorous tagged protein.

In the present embodiment, the group having the boric acid group can be a group represented by formula (1) below:

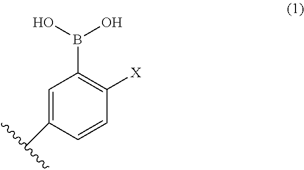

wherein X is hydrogen or a substituent containing at least one atom of nitrogen, oxygen, or sulfur. Since the group represented by formula 1 has a benzene ring, electrons adjacent to the boric acid can be stabilized.

In an embodiment of the invention, provided that X in formula 1 is hydrogen, the boric acid group of the fluorous compound can form a cyclic boronate ester with the diol group on the sugar chain of the target protein such that the fluorous compound is bonded to the target protein.

In another embodiment of the invention, provided that X in formula 1 contains the substituent of at least one atom of nitrogen, oxygen, or sulfur, an ortho position X located at the boric acid group can be used as the hydrogen bond acceptor. Moreover, the boric acid group of the fluorous compound can form an ester bond with one of the hydroxyl groups in the diol group on the sugar chain, and a hydrogen bond acceptor X of the fluorous compound can form a hydrogen bond with another hydroxyl group in the diol group on the sugar chain, such that the fluorous compound is bonded to the target protein.

### [Method of Immobilizing Protein of the Invention]

The method of preparing a fluorous tagged protein of the invention includes the following steps.

First, a fluorine-modified surface is provided. The fluorine-modified surface is, for instance, the surface of a chip or the surface of a nanoparticle, but the invention is not limited thereto.

Next, the fluorous tagged protein is brought in contact with the fluorine-modified surface, wherein the fluorous tagged protein is immobilized on the fluorine-modified surface via fluorous-fluorous interaction. Specifically, the fluorous tagged protein is immobilized on the fluorine-modified surface via the fluorous-fluorous interaction between the fluorous group and the fluorine-modified surface.

Since in the invention, immobilization on the fluorine-modified surface is achieved via the fluorous-fluorous interaction between the fluorous group of the fluorous tagged protein and the fluorine-modified surface, orientation specificity is achieved, and protein non-specific adsorption can be reduced. Therefore, the protein immobilization method of the invention is suitable for a protein chip or protein purification.

In the following, the above embodiments are described in more detail with reference to examples. However, the examples are not to be construed as limiting the scope of the invention in any sense.

### [Forming Method of Fluorine Compound of the Invention]

[Forming of Intermediate Product]

### Synthesis Example 1: Synthesis of Compound 7

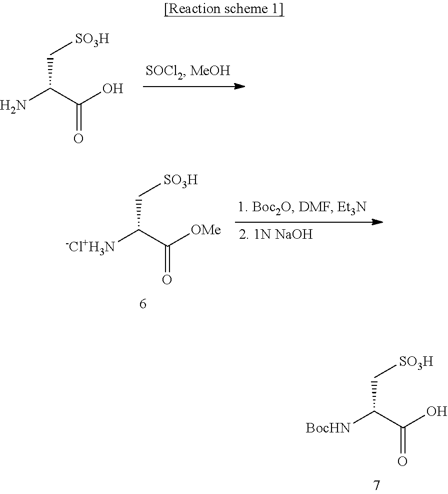

Cysteic acid (2 g, 10.7 mmol) was dissolved in anhydrous methanol (MeOH) (50 mL), and the mixture was cooled to 0° C. Next, thionyl chloride (SoCl₂) (2.3 mL, 32.1 mmol) was added to the mixture dropwise at 0° C., and the mixture was stirred at 50° C. for 3 hours. Next, the reaction solvent and thionyl chloride were removed via evaporation under reduced pressure. The residue was washed with ice acetone to obtain white solid compound 6 (2.3 g, yield: 99%).

Next, di-tert-butyl dicarbonate (Boc₂O) (3.4 g, 15.6 mmol) was added to a dimethylformamide (DMF) (50 mL) solution of compound 6 (1.9 g, 10.4 mmol) and trimethylamine (Et₃N) (3.8 mL, 27.2 mmol) at room temperature. The reaction mixture was stirred at 50° C. for 4 hours. After the raw materials were consumed, the solvent was removed under reduced pressure to obtain a crude product. The crude product (2.8 g, 10.0 mmol) was dissolved in 1N NaOH (26.0 mL, 26.0 mmol) in an ice bath. The solution was stirred at 4° C. for 3 hours and neutralized via the addition of 1N HCl aqueous solution. The solvent was removed under reduced pressure. The resulting residue was purified by P2 size-exclusion chromatography to obtain white solid compound 7 (2.3 g, yield: 80%).

### Synthesis Example 2: Synthesis of Compound 9

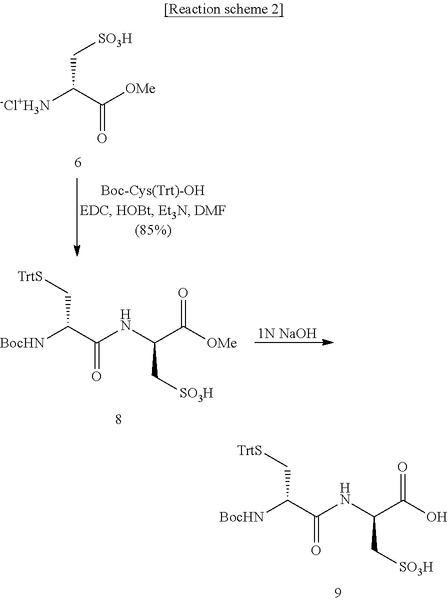

An anhydrous DMF (10.0 mL) solution of compound 6 (200 mg, 0.9 mmol), Boc-Cys(Trt)-OH (556.3 mg, 1.2 mmol), 1-ethyl-3-(3-dimethylaminopropyl)carbodiimide (EDC) (287.6 mg, 1.5 mmol), 1-hydroxybenzotriazole (HOBt) (202.7 mg, 1.5 mmol), and Et₃N (348.7 μL, 2.5 mmol) was stirred at room temperature for 12 hours. The solvent was removed under vacuum. The residue was purified via flash silica gel column chromatography (20% MeOH in an ethyl acetate/hexane (1:1) solution) to obtain compound 8 (489.7 mg, yield: 85%).

Next, compound 8 (1.2 g, 1.9 mmol) was dissolved in 1N NaOH solution (5.7 mL, 5.7 mmol) in an ice bath. The mixture was stirred at room temperature for 3 hours. Next, the resulting solution was neutralized using an HCl aqueous solution and the solvent was removed under reduced pressure. The resulting residue was purified by reverse-phase silica gel column chromatography to obtain white solid compound 9 (1.2 g, yield: 99%).

### Synthesis Example 3: Synthesis of Compound 12

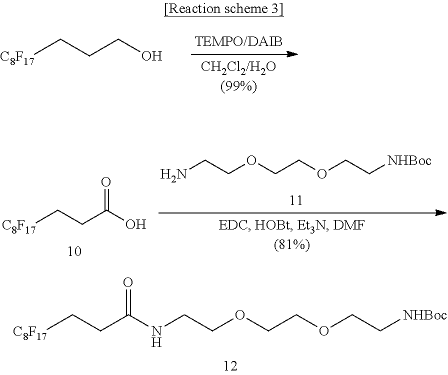

A solution of dichloromethane (DCM) (20 mL) and water (10 mL) of 3-(perfluorooctyl)propanol (2.5 g, 5.2 mmol, Fluorous Technologies Inc.), (2,2,6,6-tetramethylpiperidin-1-yl)oxyl (TEMPO) (265.6 mg, 1.7 mmol), and (diacetoxyliodo)benzene (DAIB) (4.2 g, 13.0 mmol) was violently stirred at room temperature for 5 hours. The resulting product was suspended in perfluorohexane FC-72 and washed with water and ice DCM three times to obtain compound 10 (2.2 g, yield: 90%).

Next, an anhydrous DMF (24 mL) solution of compound 1 (1.1 g, 4.4 mmol), compound 10 (1.2 g, 2.4 mmol), EDC (701.3 mg, 3.7 mmol), HOBt (494.3 mg, 3.7 mmol), and Et₃N (680.4 μL, 4.9 mmol) was stirred at room temperature for 12 hours.

The solvent was removed under vacuum. The crude product was purified via solid-phase extraction containing a fluorous solid-phase extraction cartridge. The non-fluorous compound was eluted using 80% MeOH/water and the desired product was eluted using 100% MeOH. The solvent was removed under reduced pressure to obtain white solid compound 12 (1.4 g, yield: 81%).

### Synthesis Example 4: Synthesis of Compound 13

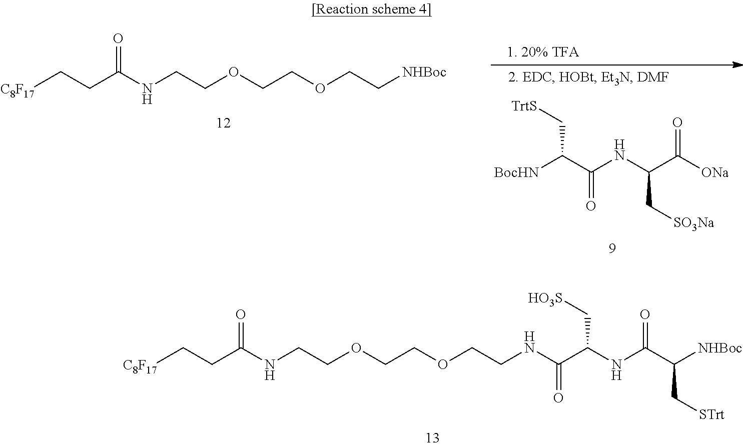

Trifluoroacetic acid (TFA) (0.8 mL) was added in a solution of compound 12 (300.0 mg, 0.4 mmol) in DCM (3.2 mL) in an ice bath. The reaction solution was stirred at room temperature for 2 hours, and then the solvent was removed under reduced pressure. An anhydrous DMF (4 mL) solution of the crude product, compound 9 (368.4 mg, 0.6 mmol), EDC (119.5 mg, 0.6 mmol), HOBt (84.2 mg, 0.6 mmol), and Et₃N (144.9 μL, 1.0 mmol) was stirred at room temperature for 12 hours, and then the solvent was removed under reduced pressure. The crude product was purified via solid-phase extraction containing a fluorous solid-phase extraction cartridge. The non-fluorous compound was eluted using 60% MeOH/water and the desired product was eluted using 100% MeOH. The solvent was removed under reduced pressure to obtain white solid compound 13 (355 mg, yield: 73%).

### Synthesis Example 5: Synthesis of Compound 14

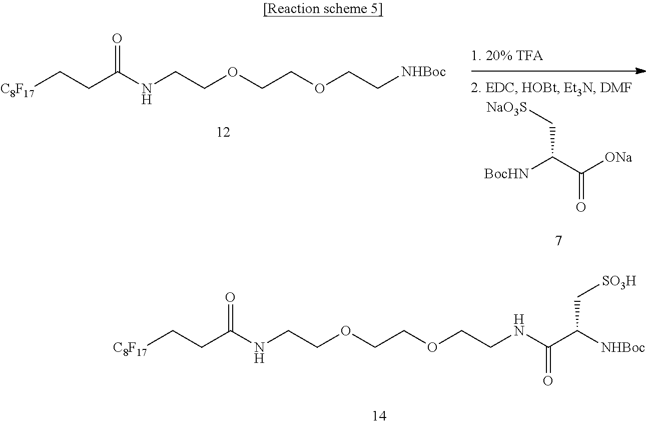

Trifluoroacetic acid (TFA) (0.8 mL) was added in a solution of compound 12 (300.0 mg, 0.4 mmol) in DCM (3.2 mL) in an ice bath. The reaction solution was stirred at room temperature for 2 hours, and then the solvent was removed under reduced pressure. An anhydrous DMF (4 mL) solution of the crude product, compound 7 (169.8 mg, 0.6 mmol), EDC (119.5 mg, 0.6 mmol), HOBt (84.2 mg, 0.6 mmol), and Et₃N (144.9 μL, 1.0 mmol) was stirred at room temperature for 12 hours, and then the solvent was removed under reduced pressure. The residue was purified via flash silica gel column chromatography (30% MeOH in an ethyl acetate/hexane (1:1) solution) to obtain compound 14 (237.8 mg, yield: 68%).

### Synthesis Example 6: Synthesis of Compound 15

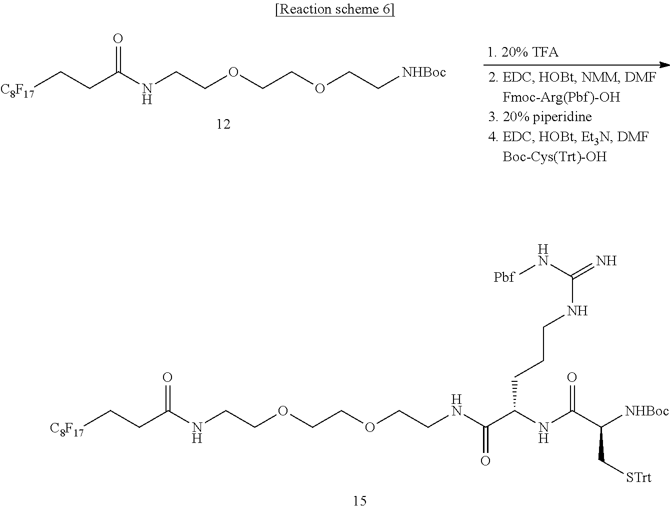

Trifluoroacetic acid (TFA) (0.8 mL) was added in a solution of compound 12 (300.0 mg, 0.4 mmol) in DCM (3.2 mL) in an ice bath. The reaction solution was stirred at room temperature for 2 hours, and then the solvent was removed under reduced pressure. An anhydrous DMF (4 mL) solution of the crude product, Fmoc-Arg(Pbf)-OH (389.3 mg, 0.6 mmol), EDC (119.5 mg, 0.6 mmol), HOBt (84.2 mg, 0.6 mmol), and N-methylmorpholine (NMM) (144.9 μL, 1.0 mmol) was stirred at room temperature for 12 hours, and then the solvent was removed under reduced pressure. The crude product was purified via solid-phase extraction containing a fluorous solid-phase extraction cartridge. The non-fluorous compound was eluted using 80% MeOH/water and the desired product was eluted using 100% MeOH. The solvent was removed under reduced pressure to obtain a white solid compound (374 mg).

Next, piperidine (0.6 mL) was added in the solution of the compound (374.0 mg, 0.3 mmol) in DMF (2.4 mL). The mixture was stirred at room temperature for 2 hours, and then the solvent was removed under reduced pressure.

Next, an anhydrous DMF (3 mL) solution of the compound, Boc-Cys(Trt)-OH (231.2 mg, 0.5 mmol), EDC (95.9 mg, 0.5 mmol), HOBt (67.6 mg, 0.5 mmol), and Et₃N (111.6 μL, 0.8 mmol) was stirred at room temperature for 12 hours, and then the solvent was removed under reduced pressure. The crude product was purified via solid-phase extraction containing a fluorous solid-phase extraction cartridge. The non-fluorous compound was eluted using 80% MeOH/water and the desired product was eluted using 100% MeOH. The solvent was removed under reduced pressure to obtain white solid compound 15 (318.7 mg, yield: 53%).

### Synthesis Example 7: Synthesis of Compound 16

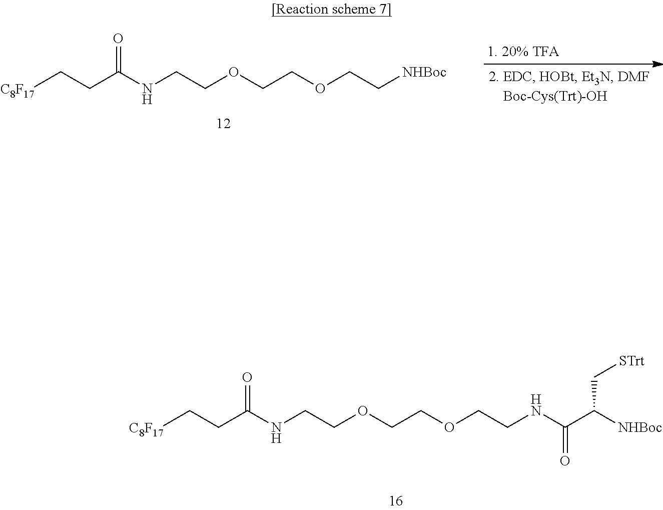

Trifluoroacetic acid (TFA) (0.8 mL) was added in a solution of compound 12 (300.0 mg, 0.4 mmol) in DCM (3.2 mL) in an ice bath. The reaction solution was stirred at room temperature for 2 hours, and then the solvent was removed under reduced pressure. An anhydrous DMF (4 mL) solution of the crude product, Boc-Cys(Trt)-OH (288.9 mg, 0.6 mmol), EDC (119.5 mg, 0.6 mmol), HOBt (84.2 mg, 0.6 mmol), and Et₃N (144.9 μL, 1.0 mmol) was stirred at room temperature for 12 hours, and then the solvent was removed under reduced pressure. The crude product was purified via solid-phase extraction containing a fluorous solid-phase extraction cartridge. The non-fluorous compound was eluted using 80% MeOH/water and the desired product was eluted using 100% MeOH. The solvent was removed under reduced pressure to obtain white solid compound 16 (305.9 mg, yield: 69%).

### Synthesis Example 8: Synthesis of Compound 17

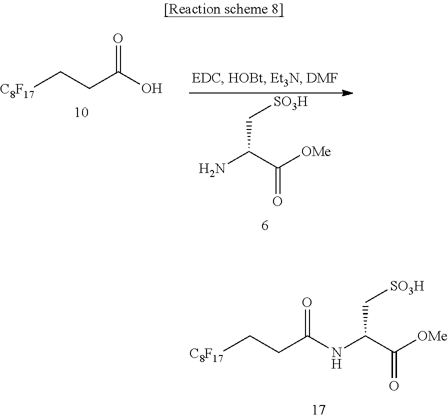

An anhydrous DMF (10.0 mL) solution of compound 10 (493.0 mg, 1.0 mmol), compound 6 (240.0 mg, 1.2 mmol), EDC (287.6 mg, 1.5 mmol), HOBt (202.7 mg, 1.5 mmol), and Et₃N (515.1 μL, 3.7 mmol) was stirred at room temperature for 12 hours. The solvent was removed under reduced pressure. The crude product was purified via flash silica gel column chromatography (20% MeOH in a DCM/acetone (1:1) solution) to obtain compound 17 (558.2 mg, yield: 85%).

### Synthesis of Final Compound

### Synthesis Example 9: Synthesis of Fluorous Compound 1

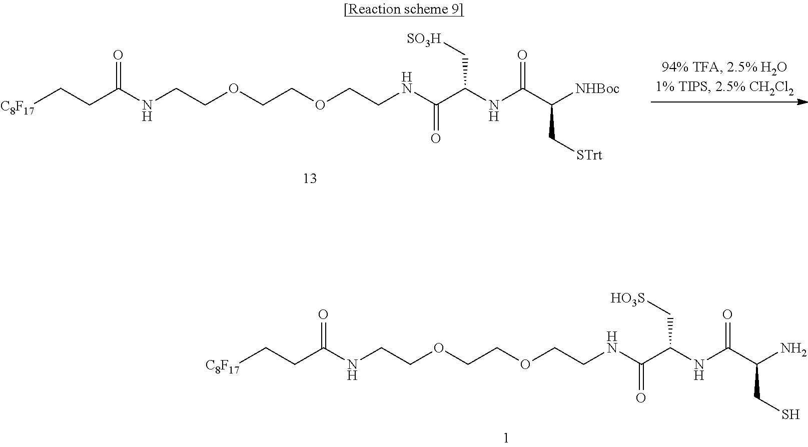

Compound 13 (100 mg, 0.01 mmol) was dissolved in a mixture (4 mL) of TFA/DCM/triisopropylsilane (TIS)/water (volume ratio: 94:2.5:2.5:1). The mixture was stirred at room temperature for 30 minutes to deprotect triphenylmethyl (Trt) and tert-butyloxycarbonyl (Boc). The solution was concentrated under reduced pressure. The resulting residue was washed with hexane and ethyl acetate to obtain fluorous compound 1 (70.9 mg, yield: 97.3%).

### Synthesis Example 10: Synthesis of Fluorous Compound 2

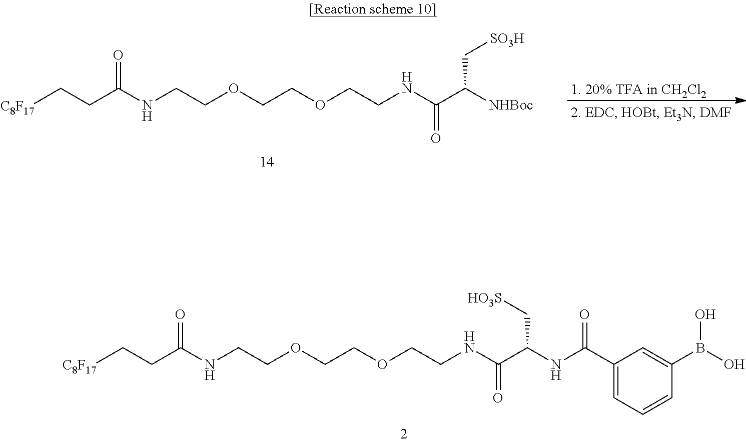

TFA (1 mL) was added to a DCM (4 mL) solution of compound 14 (430 mg, 0.5 mmol) in an ice bath. The reaction solution was stirred at room temperature for 12 hours, and then the solvent was removed under reduced pressure. Next, an anhydrous DMF (5 mL) solution of the residue, 3-carboxyphenylboronic acid (166.0 mg, 1.0 mmol), EDC (187.9 mg, 1.0 mmol), HOBt (132.4 mg, 1.0 mmol), and Et₃N (170.5 μL, 1.2 mmol) was stirred at room temperature for 12 hours. The solvent was removed under reduced pressure. The crude product was purified via silica gel chromatography (30% MeOH in an ethyl acetate/hexane (1:1) solution) to obtain fluorous compound 2 (82.9 mg, yield: 18%).

### Synthesis Example 11: Synthesis of Fluorous Compound 3

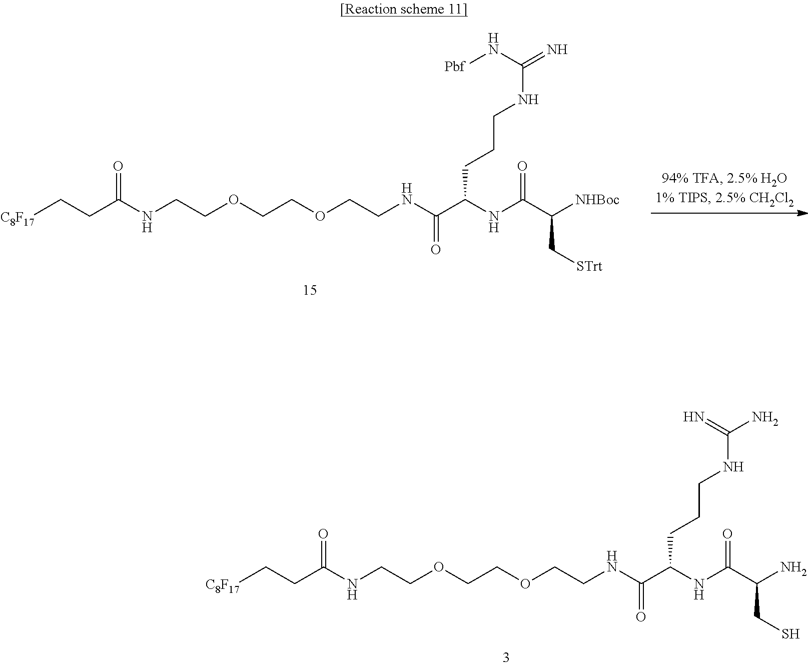

Compound 15 (100 mg, 0.1 mmol) was dissolved in a mixture (4 mL) of TFA/DCM/TIS/water (volume ratio: 94:2.5:2.5:1). The mixture was stirred at room temperature for 30 minutes to deprotect Trt and Boc. The solution was concentrated under reduced pressure. The resulting residue was washed with hexane and ethyl acetate to obtain fluorous compound 3 (56.9 mg, yield: 95.2%).

### Synthesis Example 12: Synthesis of Fluorous Compound 4

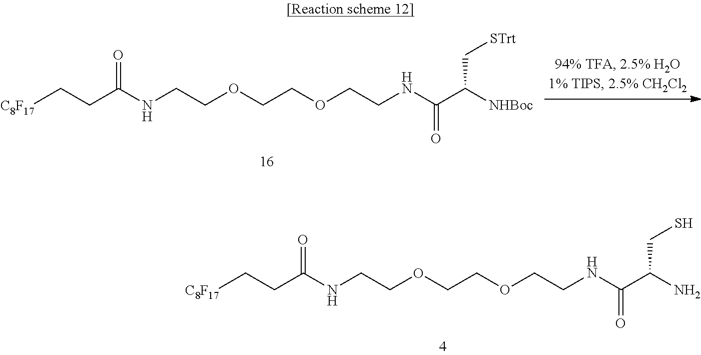

Compound 16 (100 mg, 0.1 mmol) was dissolved in a mixture (4 mL) of TFA/DCM/TIS/water (volume ratio: 94:2.5:2.5:1). The mixture was stirred at room temperature for 30 minutes to deprotect Trt and Boc. The solution was concentrated under reduced pressure. The resulting residue was washed with hexane to obtain fluorous compound 4 (65.7 mg, yield: 96.7%).

### Synthesis Example 13: Synthesis of Fluorous Compound 5

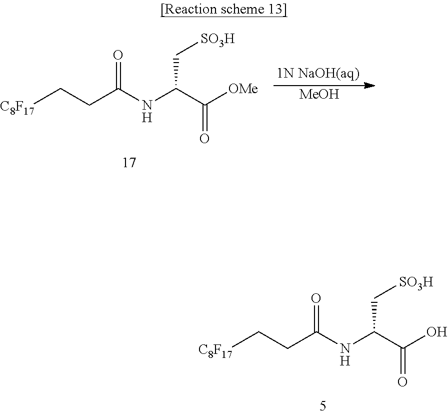

Compound 17 (660.0 mg, 1.0 mmol) was dissolved in a 1N NaOH solution (3 mL, 3 mmol) in an ice bath. The reaction solution was stirred at room temperature for 3 hours. The resulting solution was neutralized using an HCl solution, and the solvent was removed under reduced pressure. The resulting residue was purified by Toyopearl HW-40F size-exclusion chromatography to obtain white solid compound 5 (520.8 mg, yield: 81%).

[Water Solubility Test of Fluorous Compound]

Fluorous compound 1 to fluorous compound 5 (5 mg) were respectively re-dissolved by 1 mL of a 4-(2-hydroxyethyl)-1-piperazineethanesulfonic acid (HEPES) buffer solution (pH 8.0). The results are as shown in Table 1, wherein ∘ represents good water solubility and X represents poor water solubility.

TABLE 1

| Fluorous | |
|---|---|
| compound | Solubility |
| 1 | ◯ |
| 2 | ◯ |
| 3 | X |
| 4 | X |
| 5 | X |

It can be known from the content of Table 1 that, since fluorous compound 1 and fluorous compound 2 of the present application include a hydrophilic group (i.e., bivalent group of cysteic acid), the solubility of the fluorous compound to water is significantly improved, such that the efficiency of tagging protein with fluorine using the fluorous compound can be increased.

### [Preparation of Fluorous Tagged Protein]

### Example 1

In the present embodiment, MBP-intein-CBD containing maltose binding protein (MBP), intein, and chitin binding domain (CBD) was expressed by an IMPACT™-CN (Intein Mediated Purification with Affinity Chitin-binding Tag) protein expression system, wherein MBP is the target protein, and MBP and intein are connected by a thioester bond.

Next, fusion protein was reacted with 2-mercaptoethanesulfonic acid sodium salt (MESNa) to obtain MBP-MESNa for which the C segment has an activated thioester group.

Next, fluorous compound 1 (1 mM), tris(2-carboxyethyl)phosphine (TCEP) (2 mM), and MESNa (300 mM) were added in a Tris buffer solution (20 mM Tris, 500 mM NaCl, 0.1 mM EDTA, pH 8.0) containing MBP-MESNa (10 μM), and the mixture was subjected to an NCL reaction at 4° C. for 19 hours. Next, purification was performed using PD MidiTrap G-25 (Singular) to obtain F_{tag}-MBP.

### Example 2

F_{tag}-eGFP was prepared using the same method as example 1, and the difference is only that enhanced green fluorescent protein (eGFP) was used as the target protein.

### Example 3

In the present embodiment, anti-ricin antibody (anti-RCA₁₂₀) was used as the target protein of the present embodiment. A binding buffer solution (20 mM Tris, 500 mM NaCl, 0.1 mM EDTA, pH 8.0) containing fluorous compound 2 (1 mM) and anti-RCA₁₂₀ (5 μg/mL) was reacted at 4° C. for 16 hours. Next, purification was performed using PD MidiTrap G-25 (Singular) to obtain F_{tag}-anti-RCA₁₂₀.

### Comparative Example 1

Fluorine tagging of anti-RCA₁₂₀ was performed using the same method as example 3, and the difference is only that fluorous compound 5 (1 mM) and anti-RCA₁₂₀ were used for the reaction.

To verify that the fluorous tagged proteins made in example 1 to example 3 can be immobilized on the fluorine-modified surface via fluorous-fluorous interaction, experimental examples are provided below.

### [Preparation of Fluorous Tagged Magnetic Nanoparticle]

A suspension of Fe₃O₄ nanoparticles (10 mL, 56 mg/mL) was dispersed in 1-propanol (100 mL), and the resulting nanoparticle solution was treated with ultrasound for 30 minutes to disperse the aggregates. Next, 25% of NH₄OH (7.62 mL) and tetraethyl orthosilicate (TEOS) (1.87 mL) were added in the mixture. The resulting solution was stirred at 60° C. for 2 hours. Next, a mixture of 2-[methoxy(polyethyleneoxy)propyl]trimethoxysilane (mPEG) and tridecafluoro-1,1,2,2-tetrahydrooctyl)triethoxysilane (F_{tag}-(OEt)₃) (1.87 mL:0.38 mL) having a volume ratio of 5:1 was added. The resulting solution was violently stirred at 60° C. for 12 hours. Next, washing was performed using 1-propanol (5 mL×3) and water (5 mL×3) to obtain fluorous tagged magnetic nanoparticle F_{tag}-MNP on the surface.

### [Purification of Fluorous Tagged Protein]

First, the prepared F_{tag}-MNP (10 mg) was dispersed in phosphate-buffered saline with Tween-20 (5 mL) having Tween-20, and the mixture was washed with a N-(2-hydroxyethyl)-piperazine-N′-2-ethanesulfonic acid (HEPES) buffer solution (5 mL×3) and then suspended in the HEPES buffer solution (0.5 mL).

Next, the protein solution (total of 100 μg) of F_{tag}-MBP of example 1 and MBP not tagged by fluorine was added to the HEPES buffer solution containing F_{tag}-MNP, and the mixture was reacted at 25° C. for 5 minutes. Next, the F_{tag}-MNP was washed with 1 mL of the HEPES buffer solution three times to remove protein not bonded to F_{tag}-MNP.

Next, fluorous compound 5 (1 mM) of synthesis example 13 was added to the F_{tag}-MNP solution and reacted at 25° C. for 5 minutes to separate F_{tag}-MBP from F_{tag}-MNP. Via a strong magnetic force, F_{tag}-MNP was captured at the bottom of the test tube and the supernatant was recovered. Next, the supernatant was purified by PD MidiTrap G-25 (Singular) to obtain purified protein.

The purified protein and MBP not tagged by fluorine were analyzed by matrix-assisted laser desorption/ionization time-of-flight mass spectrometry (MALDI-TOF).

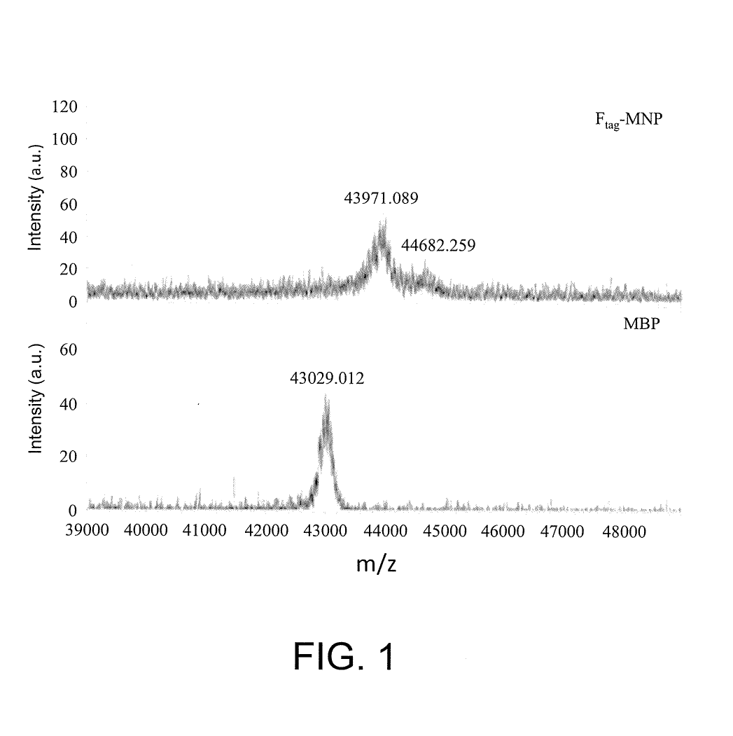

FIG. 1 is a mass spectrum of purified protein and maltose binding protein MBP not tagged by fluorine. As FIG. 1 shows, the molecular weight of the purified protein is about 43971 Da, and the molecular weight of the MBP not tagged by fluorine is about 43029 Da. The molecular weight of the purified protein is about 942 Da more than the molecular weight of the MBP not tagged by fluorine, and therefore the purified protein is verified to be F_{tag}-MBP. In other words, in the mixing process of F_{tag}-MNP and the protein solution, only F_{tag}-MBP is bonded to F_{tag}-MNP, and MBP not tagged by fluorine is not bonded to F_{tag}-MNP. It can be known from the above that, via the fluorous tagged protein prepared by the fluorous compound (i.e., fluorous compound 1) of the invention, specific immobilization can indeed occur on the surface of fluorine-modified magnetic nanoparticles via fluorous-fluorous interaction.

### [Application of Fluorous Chip]

### [Immobilization of F_{tag}-eGFP on Fluorous Chip]

### Experimental Example 1

A protein mixture of F_{tag}-eGFP (from example 2) and unmodified eGFP was prepared in a printing buffer solution (20 mM HEPES, 500 mM NaCl, and 0.1 mM EDTA, glycerin 10%), and then the mixture was dispensed on a slide (i.e., fluorous chip) having a fluorine-modified surface using a robotic contact arrayer (AD1500 Arrayer, BioDot) provided with Stealth Pins SMP3 (Arrayit). The printing process was performed at 90% relative humidity, and the temperature was kept below 26° C. The printed fluorous chip was kept in a moisturizer box and kept at 4° C. for 12 hours. Next, the fluorous chip was moved to room temperature for 2 hours to increase fluorous-fluorous interaction. Next, the fluorous chip was blocked twice using a PBS solution containing 1% of bovine serum albumin (BSA) (5 minutes each). Next, washing was performed twice with deionized water (5 minutes each) to remove non-specific adsorbed protein.

### Comparative Experimental Example 1

The target protein was immobilized on a fluorous chip using a similar method to experimental example 1, and the difference is only that the protein mixture of F_{tag}-eGFP and eGFP was replaced with unmodified eGFP protein solution.

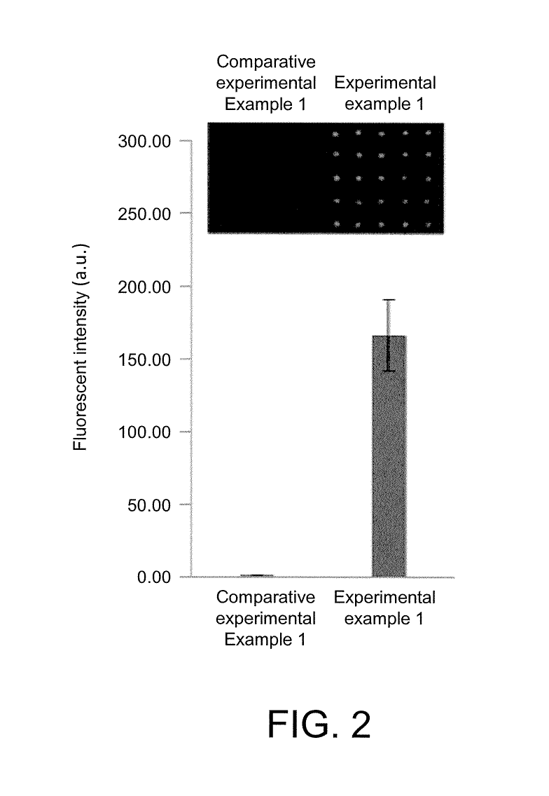

To analyze the eGFP immobilized on the fluorous chip, eGFP fluorescence activity on the fluorous chips of experimental example 1 and comparative experimental example 1 immobilized by protein was directly measured using a NovaRay microarray scanner. FIG. 2 is an eGFP fluorescence analysis chart of the fluorous chips of experimental example 1 and comparative experimental example 1 immobilized by protein. As FIG. 2 shows, the fluorous chip of comparative experimental example 1 does not detect the eGFP fluorescent signal, indicating natural eGFP cannot be immobilized on the fluorous chip. The fluorous chip of experimental example 1 can detect the eGFP fluorescent signal, indicating eGFP tagged by the fluorous compound (i.e., fluorous compound 1) of the invention can be specifically immobilized on the surface of the fluorous chip via fluorous-fluorous interaction.

### [Immobilization of F_{tag}-MBP on Fluorous Chip]

### Experimental Example 2

The target protein was immobilized on a fluorous chip using a similar method to experimental example 1, and the difference is only that the protein mixture of F_{tag}-eGFP and eGFP was replaced with a F_{tag}-MBP protein (from example 1) solution.

### Experimental Example 3

The target protein was immobilized on a fluorous chip using a similar method to experimental example 1, and the difference is only that the protein mixture of F_{tag}-eGFP and eGFP was replaced with a protein mixture of F_{tag}-MBP protein (from example 1) and MBP.

### Comparative Experimental Example 2

The target protein was immobilized on a fluorous chip using a similar method to experimental example 1, and the difference is only that the protein mixture of F_{tag}-eGFP and eGFP was replaced with an MBP protein solution.

To analyze MBP immobilized on the fluorous chips, the fluorous chips of experimental example 2, experimental example 3, and comparative experimental example 2 immobilized by protein and biotinylated anti-MBP (1 ng/μL, Vector Laboratories) were incubated at room temperature for 3 hours. After the solution was poured out, the fluorous chips were washed respectively with PBS containing 1% BSA and deionized water (5 minutes each). Next, after dyeing was performed using streptavidin-Cy3 (10 ng/μL, Sigma-Aldrich) at 4° C. for 30 minutes, the fluorous chips were washed respectively with PBS containing 1% BSA and deionized water (5 minutes each). The fluorescent signal of Cy3 was measured using a VIDAR Revolution® 4550 scanner.

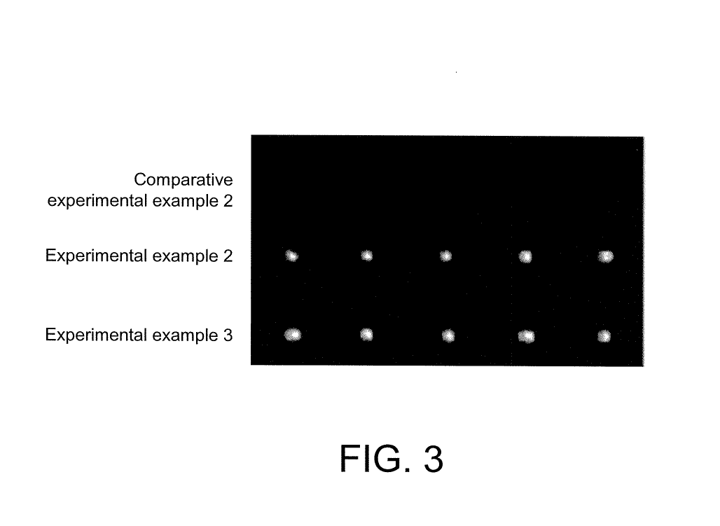

FIG. 3 is a Cy3 fluorescence analysis chart of the fluorous chips of experimental example 2, experimental example 3, and comparative experimental example 2 immobilized by protein. As FIG. 3 shows, the fluorous chip of comparative experimental example 2 does not detect the Cy3 fluorescent signal, indicating MBP not tagged by fluorine cannot be immobilized on the fluorous chip. The fluorous chips of experimental example 2 and experimental example 3 can detect the Cy3 fluorescent signal, indicating MBP tagged by the fluorous compound (i.e., fluorous compound 1) of the invention can be specifically immobilized on the surface of the fluorous chip via fluorous-fluorous interaction.

[Immobilization of F_{lag}-Anti-RCA₁₂₀ on Fluorous Chip]

### Experimental Example 4

The target protein was immobilized on a fluorous chip using a similar method to experimental example 1, and the difference is only that the protein mixture of F_{tag}-eGFP and eGFP was replaced with F_{tag}-anti-RCA₁₂₀ protein (from example 3) Solution

### Comparative Experimental Example 3

The target protein was immobilized on a fluorous chip using a similar method to experimental example 1, and the difference is only that the protein mixture of F_{tag}-eGFP and eGFP was replaced with anti-RCA₁₂₀ protein solution.

### Comparative Experimental Example 4

The target protein was immobilized on a fluorous chip using a similar method to experimental example 1, and the difference is only that the protein mixture of F_{tag}-eGFP and eGFP was replaced with anti-RCA₁₂₀ protein solution of comparative example 1 reacted with fluorous compound 5.

To analyze anti-RCA₁₂₀ immobilized on the fluorous chip, the fluorous chips of experimental example 4, comparative experimental example 3, and comparative experimental example 4 immobilized by protein and RCA₁₂₀ (1 μg/μL, Sigma-Aldrich) used as an antigen were incubated at room temperature for 2 hours. After the solution was poured out, the fluorous chips were washed respectively with PBS containing 1% BSA and deionized water (5 minutes each). Next, the fluorous chips and biotinylated anti-RCA₁₂₀ (1 ng/μL, Novus Biological) were incubated at room temperature for 3 hours. After the solution was removed, the fluorous chips were washed respectively with PBS containing 1% BSA and deionized water (5 minutes each). Next, after dyeing was performed using streptavidin-Cy3 (10 ng/μL, Sigma-Aldrich) at 4° C. for 30 minutes, the fluorous chips were washed respectively with PBS containing 1% BSA and deionized water (5 minutes each). The fluorescent signal of Cy3 was measured using a VIDAR Revolution® Revolution 4550 scanner.

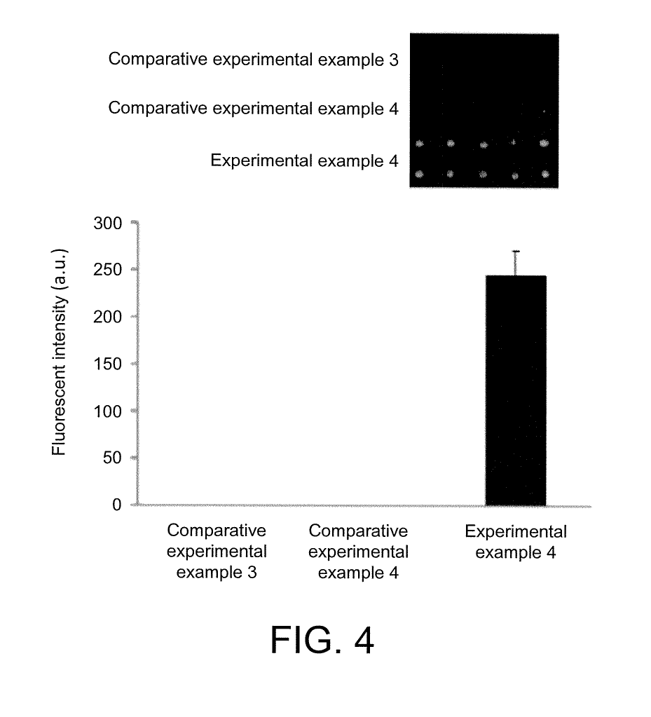

FIG. 4 is a Cy3 fluorescence analysis chart of the fluorous chips of experimental example 4, comparative experimental example 3, and comparative experimental example 4 immobilized by protein. As FIG. 4 shows, the fluorous chip of comparative experimental example 3 does not detect the Cy3 fluorescent signal, indicating anti-RCA₁₂₀ not tagged by fluorine cannot be immobilized on the fluorous chip. Moreover, the fluorous chip of comparative experimental example 4 also does not detect the Cy3 fluorescent signal because fluorous compound 5 cannot be bonded on anti-RCA₁₂₀ via boronate ester bond formation, and therefore anti-RCA₁₂₀ of comparative example 1 is not tagged by fluorine and cannot be immobilized on the fluorous chip. The fluorous chip of experimental example 4 can detect the Cy3 fluorescent signal, indicating anti-RCA₁₂₀ tagged by the fluorous compound (i.e., fluorous compound 2) of the invention can be specifically immobilized on the surface of a fluorous chip via fluorous-fluorous interaction, and the immobilization method does not compromise the bonding capacity between the antibody and the antigen.

Based on the above, since the fluorous compound of the embodiments includes a hydrophilic group, the solubility of the fluorous compound to water is significantly improved, such that the efficiency of subsequently tagging protein with fluorine using the fluorous compound can be increased. Moreover, the fluorous tagged protein is prepared using the fluorous compound of the embodiments, and therefore higher fluorous tagging efficiency is achieved. Moreover, since the method of protein immobilization of the embodiments can immobilize the fluorous tagged protein on a fluorine-modified surface via fluorous-fluorous interaction, orientation specificity can be achieved, and protein non-specific adsorption can be reduced.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A fluorous compound represented by Y-L-R, wherein Y is a fluorous group; L is a linker, and the linker comprises a bivalent group having a sulfo group, a bivalent group having a carboxyl group, or a bivalent group of a hydrophilic amino acid; and R is a functional group capable of bonding to a protein.

2. The fluorous compound of claim 1, wherein the linker further comprises a C1 to C12 alkylene group, a C6 to C15 arylene group, a C2 to C12 heteroarylene group, a C1 to C12 alkyleneoxy group, a C1 to C12 alkylene sulfide group, a C3 to C12 cycloalkylene group, an amide group, a bivalent group of ethylene glycol, or a combination thereof.

3. The fluorous compound of claim 1, wherein the bivalent group having the sulfo group is a bivalent group of cysteic acid.

4. The fluorous compound of claim 1, wherein the bivalent group of the hydrophilic amino acid comprises a bivalent group of aspartic acid, a bivalent group of glutamic acid, or a bivalent group of arginine.

5. The fluorous compound of claim 1, wherein the fluorous group comprises a straight or branched C3 to C8 fluoroalkyl group.

6. The fluorous compound of claim 1, wherein the functional group capable of bonding to the protein comprises a group having a thiol group and an amine group, a group having a thioester group, or a group having a boric acid group.

7. The fluorous compound of claim 6, wherein the group having the boric acid group is a group represented by formula (1) below: 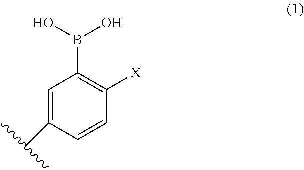
wherein X is hydrogen or a substituent containing at least one atom of nitrogen, oxygen, or sulfur.

8. The fluorous compound of claim 1, wherein the fluorous compound is a compound represented by formula (2) or a compound represented by formula (3) below:

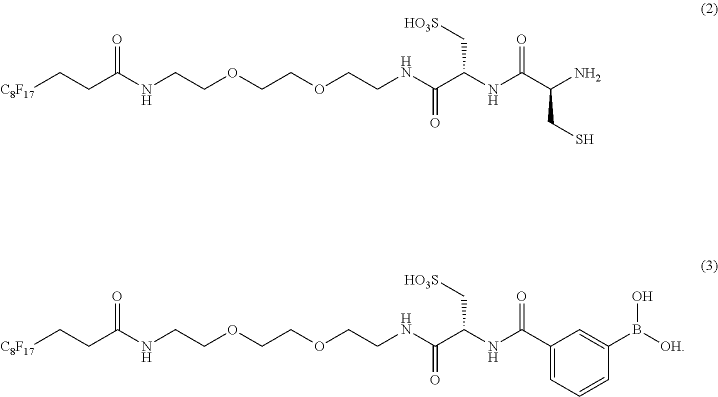

9. A method of preparing a fluorous tagged protein, comprising:
providing a protein; and
bonding a fluorous compound to the protein, and the fluorous compound is represented by Y-L-R, wherein
Y is a fluorous group; L is a linker, and the linker comprises a bivalent group having a sulfo group, a bivalent group having a carboxyl group, or a bivalent group of hydrophilic amino acid; and R is a functional group capable of bonding to a protein.

10. The method of preparing the fluorous tagged protein of claim 9, wherein the linker further comprises a C1 to C12 alkylene group, a C6 to C15 arylene group, a C2 to C12 heteroarylene group, a C1 to C12 alkyleneoxy group, a C1 to C12 alkylene sulfide group, a C3 to C12 cycloalkylene group, an amide group, a bivalent group of ethylene glycol, or a combination thereof.

11. The method of preparing the fluorous tagged protein of claim 9, wherein the bivalent group of the hydrophilic amino acid comprises a bivalent group of aspartic acid, a bivalent group of glutamic acid, or a bivalent group of arginine.

12. The method of preparing the fluorous tagged protein of claim 9, wherein the functional group capable of bonding to the protein comprises a group having a thiol group and an amine group, a group having a thioester group, or a group having a boric acid group.

13. The method of preparing the fluorous tagged protein of claim 12, wherein the group having the boric acid group is a group represented by formula (1) below: 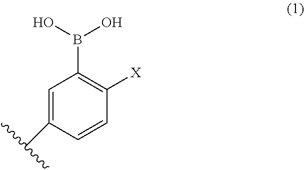
wherein X is hydrogen or a substituent containing at least one atom of nitrogen, oxygen, or sulfur.

14. The method of preparing the fluorous tagged protein of claim 12, wherein provided that the functional group capable of bonding to the protein is the group having the thiol group and the amine group, a C terminal of the protein has a thioester group, and the protein and the fluorous compound are bonded via native chemical ligation (NCL).

15. The method of preparing the fluorous tagged protein of claim 14, wherein the protein is a recombinant protein expressed using an IMPACT™-CN protein expression system.

16. The method of preparing the fluorous tagged protein of claim 12, wherein provided that the functional group capable of bonding to the protein is the group having the thioester group, an N terminal of the protein is cysteine, and the protein and the fluorous compound are bonded via NCL.

17. The method of preparing the fluorous tagged protein of claim 12, wherein provided that the functional group capable of bonding to the protein is the group having the boric acid group, the protein comprises a fragment crystallizable region (Fc region) having a sugar chain, the sugar chain comprises a diol group, and the protein and the fluorous compound are bonded by a boronate ester formation.

18. The method of preparing the fluorous tagged protein of claim 17, wherein the protein comprises a glycosylated antibody or an Fc-fusion protein.

19. A method of immobilizing a protein, comprising the following steps:
providing a fluorine-modified surface; and
bringing the fluorous tagged protein prepared by the method of preparing the fluorous tagged protein of claim 9 in contact with the fluorine-modified surface, wherein the fluorous tagged protein is immobilized on the fluorine-modified surface via a fluorous-fluorous interaction.

20. The method of immobilizing the protein of claim 19, wherein the fluorine-modified surface comprises a surface of a chip or a surface of a nanoparticle.
